(19) United States
(12) Patent Application Publication
(10) Pub. No.: US 2008/0004573 A1
(43) Pub. Date: Jan. 3, 2008

(54) Adapter for Affixing a Medical Appliance

(76) Inventors: Heiner Kaufmann, Bern (CH); Florian Kuhni, Burgdorf (CH); Axel Remde, Lutzelfluh-Goldbach (CH); Gerald Studer, Sion (CH)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/750,652

(22) Filed: May 18, 2007

(30) Foreign Application Priority Data
May 19, 2006 (CH) 815/06

(51) Int. Cl. A61M 5/32

(52) U.S. Cl. 604/180; 604/174; 604/177

(57) ABSTRACT

An auxiliary adapter with which a medical appliance carrying an adhesive can be applied to the skin of a human or animal body, wherein the adapter may be connected to or integral with the medical appliance and, in some embodiments, includes a fixing structure or portion generally correlative with a pivot mechanism associated with the medical appliance. Using the adapter, the medical appliance may be applied to the skin surface ergonomically and comfortably, and as precisely and securely as possible.

### CROSS-REFERENCE TO RELATED APPLICATION(S)

This application claims priority to Swiss Application No. 815/06, filed on May 19, 2006, which is hereby incorporated herein by reference in its entirety.

### BACKGROUND

The present invention relates to devices for dispensing, injecting, administering, infusing or delivering substances, including medicinal substances, and to methods of making and using such devices. More particularly, the present invention relates to an adapter or auxiliary device with which a medical appliance or device provided with or carrying an adhesive can be applied to the skin of a patient securely and ergonomically, and with precise positioning.

By virtue of the technical advances in microelectronics and micromechanics, it is possible to produce portable medical appliances with ever smaller structural volumes and lower inherent weights. This means that, with the advantages of enhanced wearing comfort and improved discreetness, it is possible to secure them directly to the skin surface of a human or animal body.

Portable medical appliances or devices in the form of infusion devices, e.g., pumps, that are secured directly on the skin surface and thus permit discreet and comfortable wearing, are used, for example, for treating diabetes patients. Ideally, an infusion device of this kind worn on the body adheres for 4 to 5 days to the body and is water-tight. In many instances, the infusion device is exchanged, and the location of the cannula insertion site altered, by the actual patient.

Other appliances that can be secured on the skin are known, for example, in the field of continuous recording of blood sugar measurement values in the human body. A glucose measurement sensor, mounted on a housing part remaining on the outside of the body, is introduced subcutaneously into the body tissue to determine the blood sugar value.

The securing of such medical appliances to the body has to meet a number of basic requirements to optimize a secure hold, good wearing comfort and positionally accurate placement.

For securing to the skin surface, a firm connection should be provided between the body and the medical appliance throughout the period of use. Self-adhesive plaster materials permit sufficient adherence during the period of placement of the medical appliance on the skin surface, provided that the adhesive face of the medical appliance has been contacted with and/or connected to the skin surface with sufficient surface pressure. For this purpose, an appliance that is to be affixed should be easy to handle during the application procedure. In miniaturized appliances, this requirement is often difficult to satisfy.

For secure attachment and comfortable wear, the tilting moments caused by clothing and by gravity should be kept as low as possible. This requirement can be met by a construction having the smallest possible overall height, i.e., by a device that is as thin as possible. For the manipulation and application of the medical device, however, such a configuration, which provides a reduced grip, may prove to be disadvantageous. A thicker structure or ergonomically shaped gripping features and structures, permitting secure handling of the medical appliance during the application procedure, would likely be inconvenient during the time the appliance is in use on the body.

The generally thin, flat, plate-like shape of a medical appliance designed to be secured to the skin, for example an insulin pump, means that the patient's required view of the insertion cannula, and of the sensor that is to be introduced, is obstructed during the application procedure. This makes it difficult, or even impossible, to provide for secure and positionally accurate fixing of the medical appliance to the skin surface.

In the prior art, infusion pumps that can be secured on the body are known which have a modular structure. A modular configuration of such an infusion pump is described in patent specification DE 198 21 723 C2.

Moreover, the prior art includes embodiments that disclose application or adapter devices with adhesives between medical appliance and skin surface. Patent specification WO 2004/060436 A2 shows an adapter plate with which a disposable infusion pump can be applied to the skin surface by way of differently configured adhesive surfaces. US 2004/0116866 A1 describes an infusion pump which can be adhesively affixed to the human or animal body and which has multi-part adhesive fields that permit optimal force-fit conditions at different loads. In the area of the infusion site, the use of several adhesive segments with different adhesion properties and strengths are disclosed.

### SUMMARY

An object of the present invention is to ensure that a medical appliance or device to be secured on the skin surface of a human or animal body can be applied or placed on the body manually by using an adapter device, securely and ergonomically, and with precise positioning.

In one embodiment, the present invention comprises an auxiliary adapter with which a medical appliance provided with an adhesive can be applied to the skin of a human or animal body, wherein the adapter may be connected to or integral with the medical appliance and, in some embodiments, includes a fixing portion generally correlative with a pivot mechanism associated with the appliance. Using the adapter, the medical appliance may be applied to the skin surface ergonomically and comfortably, and as precisely and securely as possible.

In one embodiment, the present invention comprises an adapter for aiding placing a medical appliance on a patient's body, the adapter comprising a gripping portion for connection to an attachment portion of the medical appliance, whereby the gripping portion facilitates placing the appliance on the body. In some embodiments, the appliance further comprises a pivot part flexibly coupled to the attachment portion, and the adapter further comprises another portion flexibly coupled to the gripping portion for connection to the pivot part, the another portion and gripping portion coupled by a fixing portion and, in some embodiments, the fixing portion is generally correlative with and/or located generally adjacent to the flexible coupling between the attachment portion and the pivot part.

In one embodiment, the present invention comprises an adapter device or auxiliary device with which a medical appliance provided with an adhesive arrangement can be applied manually to the skin surface of a human or animal body, wherein the adapter device, forming a unit with the medical appliance, is provided with a pivot mechanism and with a releasable fixing structure. The adapter device allows a medical appliance to be applied to the skin surface securely and ergonomically, and with precise positioning.

In the starting state, the adapter device is fixedly or, in some embodiments, releaseably connected to the medical appliance and is equipped with an ergonomically shaped gripping structure or portion to ensure that the miniaturized appliance can easily be held with a secure grip in the user's hand. The fixed connection between adapter device and medical appliance can be achieved both with a force fit and with a form fit. The starting state refers to the state from which the medical appliance is applied to the skin surface and in which a pivot part associated with the medical device is secured by a fixing means at a defined angle with respect to the attachment part.

The pivot mechanism divides the adapter device and the now fixedly connected medical appliance into an attachment part and a pivot part. In some embodiments, the pivot mechanism reflects that the medical device and adapter have two portions. The attachment part is that part that is first applied and affixed to the skin surface. In the starting state, the pivot part extends at an angle, in a deflected position, from the attachment part, in such a way that the adhesive on underside of the pivot part does not come into contact with the skin surface. The pivot mechanism located between attachment part and pivot part permits a movement from the deflection position to the use position. In the use position, the attachment part and pivot part lie approximately planar on the body surface and provide a secure attachment of the medical appliance via the adhesive surface connections.

One embodiment of the present invention assumes a deformability of the medical appliance. If this is not provided, for example in appliances with a rigid or flexurally stiff housing, the required flexibility has to be provided structurally, for example by an articulated connection or link between attachment part and pivot part.

The grip structure, which is situated distally on the attachment part, permits ergonomic and firm handling of the medical appliance. Its ergonomic design means that the adhesive surface can be better pressed onto the skin surface, and the necessary surface pressure for a secure adhesive connection can be maximized.

In one preferred embodiment, the pivot mechanism is in the form of an articulated connection which can be locked in a deflected position and can be released manually via a release mechanism. In this way, the adapter can be packed and sold in a space-saving manner. Before application to the skin tissue, the pivot part is bought to the deflection position, in which it locks. The pivoting to the use position is achieved by actuating the manual release mechanism or, in the case of a predetermined breaking point, by exceeding an ultimate moment generated manually on the pivot mechanism.

In another preferred embodiment, the pivot is designed as an articulated fixing connection generally between the application part and pivot part of the medical device with which the adapter is being used. The fixing connection, comprising a suitable linking structure, is constructed as or includes a predetermined breaking point or fracture threshold which permits the desired pivoting movement after a sufficient manually generated ultimate moment has been generated.

In another preferred embodiment, the predetermined breaking point can be realized in the form of a connection that is easy to release manually, for example a transverse web or bracket between the pivot part and the application part.

Another preferred embodiment, in which the articulated connection can be fixed in a locked position and is released after a moment is exceeded, permits folding or movement from any desired starting position to the in use position.

The grip section or structure associated with, connected to or integral with the adapter allows, during the securing procedure, a small pump to be held firmly in the hand, thus facilitating the insertion of a needle or cannula associated with the pump.

To provide the medical appliance with a shape that is as flat as possible, the grip section or portion may be removed from the medical appliance after application (i.e., after the appliance is placed on the body) or is designed such that the grip section or portion can be detached or removed, in whole or in part, from the housing of the appliance after use.

In another preferred embodiment, a medical appliance or medical device with a sensor device, with which a sensor element is introduced into the body tissue, is secured on the body.

### BRIEF DESCRIPTION OF THE DRAWINGS

FIG. 1 shows, in accordance with the present invention, an embodiment of an adapter in conjunction with a representative medical appliance.

FIG. 2 shows a medical appliance wherein an attachment part of a medical appliance has been affixed to the body.

FIG. 3 shows a medical appliance and an adapter in accordance with the present invention in a use position after being folding down from a deflection position (cf. FIG. 2.).

FIG. 4 shows a medical appliance in the form of an insulin pump in a use position after removal of the adapter in accordance with the present invention.

FIG. 5 shows an embodiment of an adapter in accordance with the present invention.

FIG. 6 shows an embodiment of the present invention wherein there are adhesives on the underside of a medical appliance.

FIG. 7 shows an embodiment of the present invention wherein a one-piece adhesive extends across the underside of a medical appliance.

### DETAILED DESCRIPTION

With regard to making, attaching or connecting components of the present invention and/or making the invention as a whole, unless specifically described as otherwise, conventional mechanical fasteners and methods may be used. Other appropriate fastening or attachment methods include adhesives, welding and soldering, the latter particularly with regard to the electrical system of the device, if any. In embodiments with electrical features or components, suitable electrical components and circuitry, wires, chips, boards, microprocessors, inputs, outputs, displays, control components, etc. may be used. Generally, unless otherwise indicated, the materials for making the adapter and/or the medical appliance or device of the present invention and/or their components may be selected from appropriate materials such as metal, suitable adhesives, metallic alloys, plastics, etc.

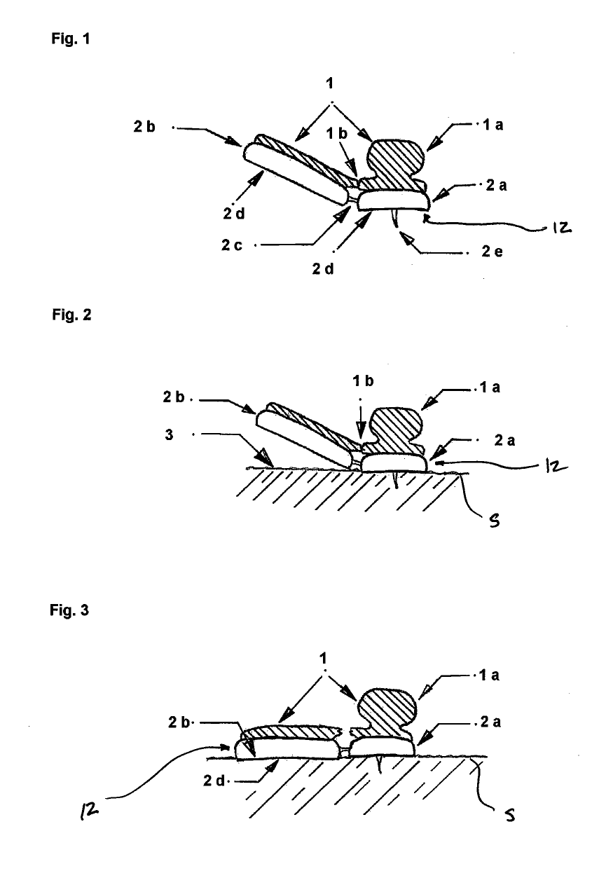

FIG. 1 shows an embodiment, according to the invention, of an adapter 1 mounted on or coupled to a medical appliance 12 comprising two portions or components 2a, 2b. In this illustrative embodiment, the medical appliance is an insulin pump designed to be secured on the skin (“S” in FIGS. 2-5) and which has an integrated catheter head. The grip or gripping portion 1a of the adapter 1 is arranged or positioned generally distally over the attachment part 2a, and allows the patient to handle the device and introduce the insertion cannula 2e into the body tissue ergonomically and with a secure hold. By virtue of the bending or deflection of the pivot part 2b relative to the skin S, the deflection being caused, enhanced and/or supported by the adapter 1, the user is afforded a good view of the insertion site, which permits positionally accurate fixing. By virtue of the good handling characteristics, i.e., a secure grip, and the raised and fixed starting position of the pivot part 2b, the attachment part 2a can be pressed securely onto the skin S.

FIG. 2 shows an infusion pump 12 in which the attachment part 2a has been affixed to the body. From this state, the pivot part 2b is pivoted or moved inwardly or downwardly toward the skin S to a use position by manually breaking an articulation or fixing structure 1b of the adapter 1 which, in the present case, is constructed as a predetermined breaking point, i.e., is designed to fracture at a certain force level.

FIG. 3 shows the infusion pump 12 and adapter 1 in a use position after the pivot part 2b has been moved from the deflection position into a position wherein both portion of the appliance 12 and the adapter 1 are generally parallel to the skin S. Attachment part 2a and pivot part 2b lie approximately planar on the skin S and are connected to the skin S by an adhesive and/or adhesive surfaces.

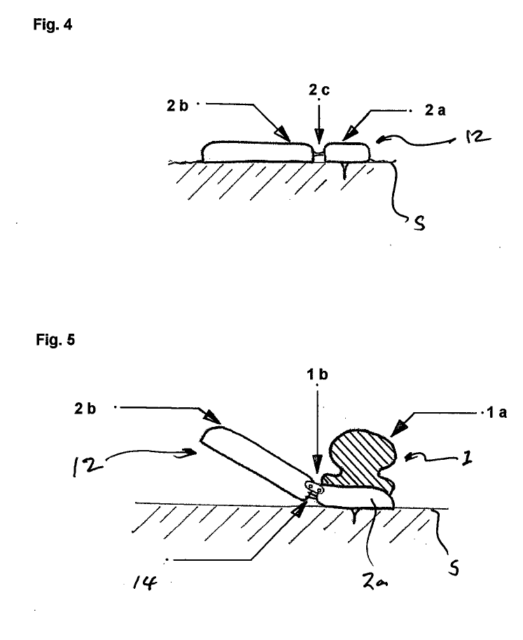

FIG. 4 shows a medical appliance in the form of an insulin pump 12 in a use position after removal of the adapter. The pump parts 2a, 2b are connected flexibly to one another by an articulated connection 2c and are adapted to closely contact and/or adhere to the skin S. In some embodiments, the articulated connection 2c may comprise a short liquid channel, integrated within or associated with the articulated connection.

FIG. 5 shows an embodiment, according to the invention, of an adapter 1 with a medical appliance 12 comprising a pivot mechanism 14 integrated with or attached to the medical appliance 12. The fixing structure 1b with a predetermined breaking point is arranged between pivot part 2b and attachment part 2a. As shown, the adapter 1, in some embodiments of the present invention, can be designed as one piece, with the grip part 1a distally over the attachment part 2a. In such embodiments, the adapter section of the previous embodiment, i.e., the portion 2b of the adapter 1 overlying or attached to the pivot part 2b of the medical device 12 is dispensed with.

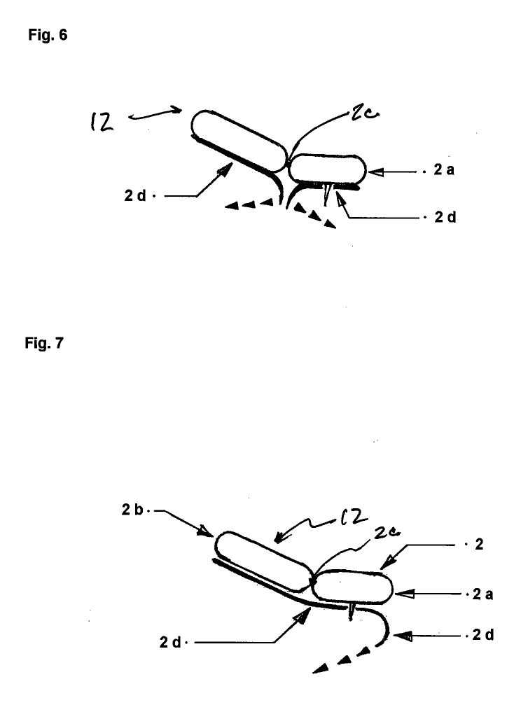

FIG. 6 shows an embodiment of a medical device 12 with adhesives 2d on the underside, in this embodiment, on both pivot part 2b and attachment part 2a. It should be appreciated that the articulated configuration of the medical appliance 12 with the pivot mechanism 2c ensures secure, conforming application to a non-planar skin surface or portion of a patient's body.

FIG. 7 shows another embodiment of a medical device 12 wherein a single adhesive portion or structure 2d, e.g., one-piece sheet or layer of a suitable adhesive, extends across the underside of the medical appliance 12.

It should be understood that any embodiment of a medical appliance in accordance with the present invention, e.g., the embodiments depicted in FIGS. 5-7 could be used in conjunction with an embodiment of the adapter 1 of the present invention.

Embodiments of the present invention, including preferred embodiments, have been presented for the purpose of illustration and description. They are not intended to be exhaustive or to limit the invention to the precise forms and steps disclosed. Obvious modifications or variations are possible in light of the above teachings. The embodiments were chosen and described to provide the best illustration of the principles of the invention and the practical application thereof, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. An adapter with which a medical appliance comprising an attachment portion and an adhesive arrangement whereby the appliance can be applied to the skin of a patient, wherein the adapter forms a unit with the medical appliance in an initial state, and comprises a grip portion and comprises a grip portion and a pivot mechanism.

2. The adapter according to claim 1, wherein the medical appliance further comprises a pivot portion and the pivot mechanism of the adapter is generally between the attachment portion and the pivot portion.

3. The adapter according to claim 2, wherein the pivot mechanism permits a deflection of the pivot portion to a deflected position which, when the attachment portion is applied to the skin, prevents an adhesive contact between the pivot portion and the skin.

4. The adapter according to claim 3, wherein the pivot mechanism comprises at least one fixing means for releaseably fixing the position of the pivot portion and for, after the fixing means has been released, allowing the pivot portion to be moved from the deflected position to a use position.

5. The adapter according to claim 4, wherein the pivot mechanism is a hinge having a locked position and forming the fixing means.

6. The adapter according to claim 4, wherein the fixing means comprises a predetermined breaking point which permits a pivoting movement from the deflected position to the use position after a torque acting on the pivot mechanism is exceeded.

7. The adapter according to claim 4, wherein the fixing means comprises a predetermined breaking point which is severed when a shearing force acting on the predetermined breaking point which, when exceeded, enables a pivoting movement from the fixed deflected position to the use position.

8. The adapter according to claim 4, wherein the fixing means comprises a removable web which, after it has been removed, enables a pivoting movement of the pivot portion from the fixed deflected position to the use position.

9. The adapter according to claim 8, wherein the adapter, in the starting state, is connected with a force-fit to the medical appliance.

10. The adapter according to claim 8, wherein the adapter can be at least partially removed from the medical appliance after a securing procedure is completed.

11. The adapter according to claim 8, wherein the grip portion can be detached from the medical appliance after a securing procedure is completed.

12. The adapter according to claim 1, wherein the pivot mechanism is integrated in the adapter if the medical appliance exhibits low flexural strength.

13. The adapter according to claim 1, wherein the pivot mechanism is integrated in the medical appliance.

14. The adapter according to claim 2, wherein the adhesive arrangement comprises an adhesive applied to an underside of the medical appliance, on the attachment and pivot portions, in the form of adhesive strips wherein the adhesive strips can be removed independently.

15. The adapter according to claim 2, wherein the adhesive arrangement comprises an adhesive are applied to an underside of the medical appliance, on the attachment and pivot portions in a contiguous adhesive strip.

16. The adapter according to claim 1, wherein the medical appliance is an administration device which can be secured on the body of the patient and which is used for medical administration of a liquid substance.

17. The adapter according to claim 1, wherein the medical appliance is an insulin pump that can be secured on the human body.

18. The adapter according to claim 1, wherein the medical appliance is a device which can be secured on the skin and which has at least one sensor for determining at least one physical condition.

19. An adapter for aiding placing a medical appliance on a patient's body, said adapter comprising a gripping portion for connection to an attachment portion of the medical appliance, whereby the gripping portion facilitates placing the appliance on the body.

20. The adapter according to claim 19, wherein the appliance further comprises a pivot part flexibly coupled to the attachment portion, and the adapter further comprises another portion flexibly coupled to the gripping portion for connection to the pivot part, said another portion and gripping portion coupled by a fixing portion.

21. The adapter according to claim 20, wherein the fixing portion is generally correlative with the flexible coupling between the attachment portion and the pivot part.
